(19) United States
(12) Patent Application Publication
(10) Pub. No.: US 2008/0262451 A1
(43) Pub. Date: Oct. 23, 2008

(54) Device for Collecting Urine or Other Organic Body Fluids, a Laminate Sheet and a Method for Producing Said Sheet

(76) Inventor: Bengt-Inge Broden, Skara (SE)

Correspondence Address: HOLLAND & HART, LLP, P.O BOX 8749, DENVER, CO, 80201, US

(21) Appl. No.: 11/575,560

(22) PCT Filed: Oct. 11, 2005
(86) PCT No.: PCT/SE2005/001505

§ 371 Date: Aug. 13, 2007

(30) Foreign Application Priority Data
Oct. 12, 2004 (SE) 0402468-3

(51) Int. Cl. A61F 5/451

(52) U.S. Cl. 604/347

(57) ABSTRACT

The present invention relates to a device for collecting urine or other organic body fluids. The device comprises a flexible bag (1) made of liquid-impermeable film material, a member (2) for receiving fluid and conveying it into the bag, means for preventing fluid from leaving the bag via said receiving member, and at least one absorption body arranged in the bag (1) and in the form of a sheet-shaped laminate (5). The laminate sheet comprises a superabsorbent material which is enclosed between two liquid-permeable surface layers and which forms a gel composition (14) when delivered fluid is absorbed. The surface layers of the laminate sheet (5) have such a porosity that delivered fluid in brief contact with a surface layer of a non-horizontal sheet (5) will run down the outside of the latter without penetrating to any appreciable extent through the surface layer, whereas delivered fluid which forms a fluid collection in contact with a surface layer of the sheet penetrates through the latter so as to be absorbed by the superabsorbent material located to the inside of the surface layer and so as to convert this superabsorbent material to a gel composition (14).

### TECHNICAL FIELD

The present invention relates to a device for collecting urine or other organic body fluids, comprising a flexible bag made of liquid-impermeable film material, a member for receiving fluid and conveying it into the bag, means for preventing fluid from leaving the bag via said receiving member, and at least one absorption body arranged in the bag and in the form of a sheet-shaped laminate comprising a superabsorbent material which is enclosed between two liquid-permeable surface layers and which forms a gel composition when delivered fluid is absorbed.

The invention also relates to a laminate sheet included in the device, and to a method for producing such a laminate sheet.

### BACKGROUND TO THE INVENTION

A device of the kind specified in the introduction eliminates, among other things, many of the problems associated with the previous use of bottles and bedpans by patients confined to wheelchairs and patients who are bedridden. Furthermore, the device improves hygiene both for patients and nursing personnel, and much of the unpleasantness involved in handling bedpans can be eliminated, because the urine is handled while enclosed in liquid-impermeable plastic bags that are intended to be discarded.

Collection devices of this kind can also be used by healthy people in many circumstances, for example in automotive vehicles, boats and aircraft, and by persons who spend long periods of time in other enclosed spaces or are not able to leave a position, for example when on guard duty.

A device of the type mentioned above, but without superabsorbent material, is described in my European patent EP 0 772 430.

To avoid handling bags filled with fluid, such as urine, it has previously been proposed to place a superabsorbent material in collection bags. When urine, for example, is delivered to such a bag, the superabsorbent material, upon absorption of the delivered urine, will be converted to a gel composition, which has several advantages in terms of the use of the bag and its subsequent handling.

U.S. Pat. No. 6,186,990 describes a urine-collecting bag which comprises a superabsorbent material enclosed in a liquid-permeable envelope. All the superabsorbent material in the liquid-permeable envelope is exposed in its entirety upon each delivery of new urine to the bag. This means, for example, that the superabsorbent material in the urine-collecting bag will work effectively when urine in normal amounts is delivered a first time and perhaps a second time, whereas upon further delivery of urine a liquid mixture is obtained which is unable to bind the newly delivered urine.

GB 2301350 A describes a method for producing a superabsorbent product which can have the shape of a laminate sheet comprising two surface layers and a superabsorbent material bound between these. EP 1177781 A2 describes the use of a superabsorbent body in the form of a laminate sheet which is said to increase the amount of urine that can be absorbed and bound in the form of a gel in a urine-collecting bag.

According to the last-mentioned patent specification, the bag is designed with at least two chambers, the urine being delivered to a first receiving chamber from which it flows across to a second collecting chamber in which the superabsorbent laminate sheet is arranged. To prevent urine from coming into contact with the upper part of the laminate sheet, the upper part of the collecting chamber is separated from the receiving chamber by a liquid-impermeable wall. Moreover, the laminate sheet is intended to be arranged on, for example bonded to, a wall of the bag so that it is at all times upright and extends over the entire height of the collecting chamber. This known arrangement is relatively complicated to produce since it requires a number of extra production stages for forming an intermediate wall with a liquid-impermeable part and a liquid-permeable part, and requires the arrangement of the laminate sheet such that the latter is constantly maintained upright.

### OBJECT OF THE INVENTION

It is an object of the present invention to provide a urine-collecting device of the kind mentioned in the first paragraph, which device, despite comprising an effective superabsorbent material, is simple and inexpensive to produce and easy to use and can be handled hygienically after use.

Another object is to make available a simple and cost-effective method of producing a superabsorbent laminate sheet included in a device according to the invention.

The invention is based on the recognition that the first of the abovementioned objects can be achieved by designing the laminate sheet such that its surface layers themselves prevent delivered fluid in brief contact with the surface layer from penetrating through the latter, but fluid in contact with the surface layer is rapidly conveyed through this. Such a laminate sheet can be placed loosely in a urine-collecting bag without the need for any intermediate wall protecting the upper part of the sheet from contact with the delivered urine.

To ensure that the correct amount of superabsorbent material is always used for absorption of delivered urine for converting the latter to a gel composition, the laminate sheet according to the invention must be allowed to collapse or “break up” from its lower end upward at the rate at which the urine to be converted to gel is delivered. Thus, no more superabsorbent material is used than is actually necessary on each occasion for conversion of the actual amount of urine, and this means an optimal utilization of the available superabsorbent material in the bag.

The special characteristic of a device of the type mentioned in the first paragraph is that the surface layers of the laminate sheet have such a porosity that delivered fluid in brief contact with a surface layer of a non-horizontal sheet will run down the outside of the latter without penetrating to any appreciable extent through the surface layer, whereas delivered fluid which forms a fluid collection in contact with a surface layer of the sheet penetrates through the latter so as to be absorbed by the superabsorbent material located to the inside of the surface layer and so as to convert this superabsorbent material to a gel composition.

In a preferred embodiment of a device according to the invention, the laminate sheet is intended to be supported by the bottom of the bag, the surface layers of the sheet being made of a flexible material which allows the laminate sheet to collapse toward the bottom of the bag as the superabsorbent material in the sheet is converted to a gel composition by the delivered fluid.

The surface layers of the laminate sheet are preferably made of compressed tissue material. This material has such a pore size that fluid in brief contact with a surface layer does not pass through the latter.

According to the invention, the superabsorbent laminate sheets are produced by two layers of tissue material and one intermediate layer of superabsorbent material being rolled together to form a blank from which laminate sheets of the desired size are punched out. A roll pressure is used which gives the blank such stiffness that the laminate sheets punched out from it are self-supporting and can be placed on the bottom of a collection bag intended for receiving urine or other organic body fluids, and which roll pressure compresses the tissue material in the surface layers of the laminate sheets in order to reduce the pore size of these to such a degree that fluid in brief contact with a surface layer does not penetrate to any appreciable extent through the latter.

The tissue material in the surface layers of the laminate sheets is expediently compressed to a density of 0.7 g/cm³±20%, preferably 0.7 g/cm³±10%, and most preferably 0.7 g/cm³±5%.

On application of the above-described process, the laminate sheet can be produced such that the tissue material in the surface layers constitutes 2-6 percent by weight, preferably ca. 3 percent by weight of the laminate sheet.

It is preferable that the surface layers of tissue material are compressed to such a degree that they acquire a pore size of 200 μm+100 μm, preferably 250 μm±50 μm.

To ensure that loose superabsorbent material in the laminate sheet does not become detached and settle on areas of the collection bag which are to be welded together during the latter's production, something which may lead to weld seams that are not completely leaktight, it is preferable that at least the side edges of the punched-out laminate sheets are moistened so that the superabsorbent material located between the edge portions of the surface layers is converted to a gel strand which closes the side edges. The moistening is expediently done with the aid of water vapor.

In another preferred embodiment, the superabsorbent material in the laminate sheet is divided into mutually separate sections, from the lower end portion of the sheet upward. This can be done by the surface layers being joined and bonded to one another along transverse connection lines. The result of this is that fluid delivered to the bag converts the superabsorbent material to a gel composition in one section after another, starting in the section located at the bottom of the bag. Thus, superabsorbent material is used up in only so many sections as are necessary on each occasion, which means optimal utilization of the superabsorbent material.

Other features of the invention will become apparent from the patent claims.

The invention will be described in more detail below with reference to the illustrative embodiments shown in the accompanying drawings.

### BRIEF DESCRIPTION OF THE FIGURES

FIG. 1 is a perspective view of a device according to the invention.

FIG. 2 illustrates a device according to the invention after it has received a first quantity of urine.

FIG. 3 is a perspective view of an embodiment of a superabsorbent laminate sheet which can be included in the device according to FIGS. 1 and 2.

FIG. 4 is an enlarged detail through an edge portion of the laminate sheet in FIG. 3.

FIG. 5 illustrates a second embodiment of a device according to the invention.

### DETAILED DESCRIPTION OF THE PREFERRED EMBODIMENTS

The collecting device according to FIG. 1 comprises a liquid-impermeable plastic film which has been folded and welded together to form a bag 1. A funnel-shaped receiving member 2, intended to receive urine and convey it into the bag, is fitted near one short side of the substantially rectangular bag 1. This facilitates use of the bag, even by a bed-ridden patient. The upper contour of the funnel is precisely shaped to allow the device to be placed in a leaktight manner against the skin when used by women.

To make it easier to press the funnel against the skin, it is provided with a handle 3. To fit the funnel on the bag 1, the bag is provided with a mounting flange 4 in which the funnel 2, by virtue of its conical shape, can be pressed down to provide a liquid-tight abutment against the flange 4. Two substantially semicircular cuts (not shown) are formed within the area of the upper film sheet of the bag 1 enclosed by the flange 4. These cuts form two flaps which can cooperate with a bottom surface of the funnel to produce a nonreturn valve function. This is described in more detail in my abovementioned European patent EP 0 772 430. The funnel with the bag can be designed entirely in the manner described in said patent, which is incorporated by reference in the present application. The fitting of the funnel on the bag and said nonreturn valve function will not be described in any more detail here.

Reference number 5 designates a superabsorbent laminate sheet arranged in the bag 1. This sheet comprises (see enlarged section in FIG. 1) two surface layers 6 and an intermediate layer 7 of superabsorbent material, expediently in powder form with a particle size of less than 600 μm. The superabsorbent material used can be crosslinked sodium polyacrylate or another material having superabsorbent properties, for example one of those specified in GB 2325432 A. The important point is that the superabsorbent material must have extremely high absorbency and, upon absorption of fluid such as urine, must be converted rapidly to a gel composition.

The surface layers 6 are made of tissue material produced from pure bleached cellulose which is compressed to give very thin, flexible, gauze-like layers with no inherent stiffness. The superabsorbent laminate sheet is produced by two or more layers of tissue material and one intermediate layer of superabsorbent material being rolled together to form a blank from which laminate sheets of the desired size are punched out. This rolling-together can in principle be carried out in the manner illustrated in GB 2301350 A.

According to the present invention, a roll pressure is used which gives the blank such stiffness that the laminate sheets punched out from it are self-supporting and can be placed on the bottom of a collection bag intended to receive urine or other organic body fluids. The roll pressure is also chosen such that the tissue material in the surface layers of the laminate sheets is compressed to such an extent that the desired porosity thereof is achieved. The desired porosity is determined by which fluid is to be absorbed by the laminate sheet. The porosity must be such that the fluid in question, in brief contact with a surface layer of a non-horizontal sheet, will run down the delivered to the bag 1 via the funnel 2 and has run down along the surface layers of the superabsorbent laminate sheet 5. Because the porosity of the surface layers has been chosen in accordance with the above, no appreciable amount of the delivered urine has passed through the surface layers. The delivered urine has gathered at the bottom of the bag and has formed a fluid collection around the lower part of the laminate sheet. The urine in this fluid collection has been able to pass rapidly through the pores in the surface layers of the laminate sheet and has converted the intermediate superabsorbent material to a gel composition 14. The lower part of the laminate sheet 5 has collapsed toward the bottom of the bag 1 or been broken up. In accordance with the above, the surface layers 6 have no inherent bearing capacity when the superabsorbent material has been converted to a gel together with the urine.

The part of the sheet 5 whose superabsorbent material has not been needed for the absorption of the delivered amount of urine and for the gel formation is still substantially intact in the bag 1 above the gel composition 14. When the next quantity of urine is delivered in conjunction with the next discharge, another part of the sheet 5 is used, and so on until the entire sheet is broken up. By virtue of the fact that the sheet 5 is used only successively for the gel formation, a device according to the invention affords a very high overall capacity when the superabsorbent material is used optimally. This is also important from the point of view of cost.

When the whole laminate sheet 5 has been used up and the bag is substantially filled with a gel composition, the whole bag can be disposed of. By virtue of the nonreturn valve, this can be done without any risk of leakage. Moreover, it is more pleasant for the user if, between passing urine, he or she can keep a bag filled outside of the latter without penetrating to any appreciable extent through the surface layer, whereas delivered fluid which forms a fluid collection in contact with a surface layer of the sheet penetrates through this so as to be absorbed by the superabsorbent material located to the inside of the surface layer and so as to convert this superabsorbent material to a gel composition. The appropriate rolling pressure will depend on the number and nature of the tissue material layers and can be determined by the skilled person through tests using several different roll pressures and through checks of how the obtained sheet reacts with the intended fluid.

For a superabsorbent laminate sheet intended to be used in a bag for collecting urine, it has been found expedient to compress the tissue material to such an extent that the pore size obtained is ca. 200 μm±100 μm, preferably 250 μm±50 μm. In a laminate sheet with surface layers having such a pore size, no appreciable amount of the urine running along the sheet will penetrate through the surface layer. By contrast, urine in a collection of fluid surrounding such a sheet will rapidly penetrate through the surface layers and be absorbed by the superabsorbent material located to the inside of these. The amount of superabsorbent material in a sheet can be 30 g±5 g, preferably 28 g±3 g. The above-described compression of the tissue material in the surface layers of the laminate sheets can be expediently adapted so that these acquire a density of 0.7 g/cm³±20%, generally 0.7 g/cm³±10%. The tissue material can constitute 2-6 percent by weight, preferably ca. 3 percent by weight of the laminate sheet, whose thickness can be 2 mm.

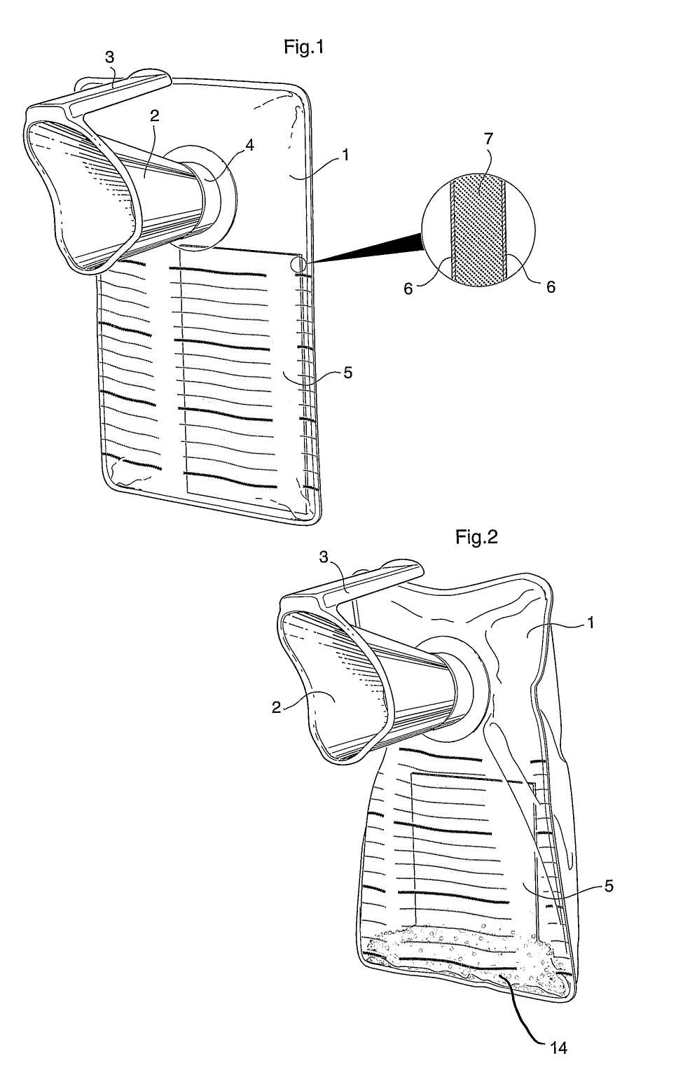

FIG. 2 shows the device according to FIG. 1 after the bag 1 has received a quantity of urine corresponding to a normal discharge of urine. The urine has been partially with gel rather than a bag which is partially filled with fluid.

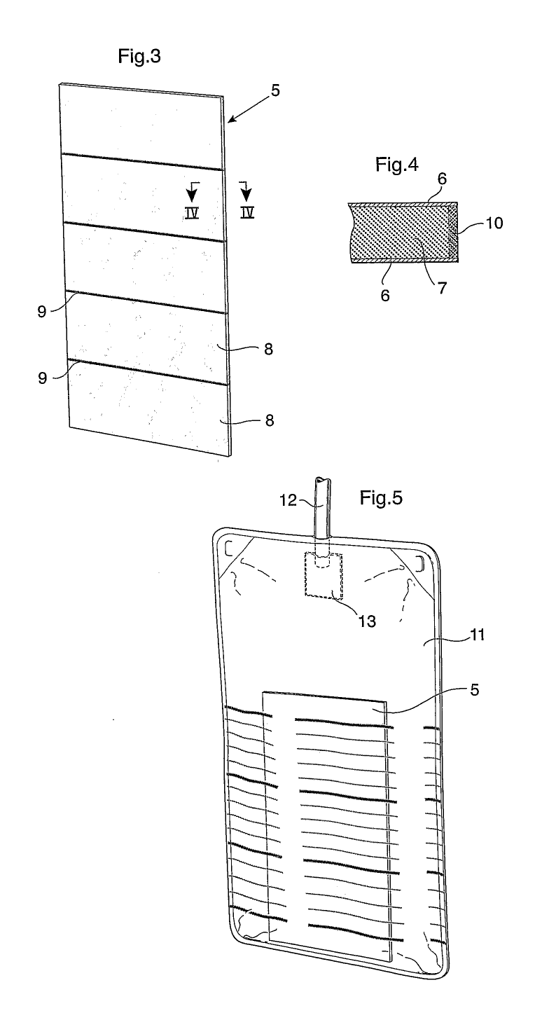

FIGS. 3 and 4 show a further development of a superabsorbent laminate sheet 5 according to the present invention. In this embodiment, the laminate sheet 5 is divided into a number of sections 8 from the lower end portion of the sheet upward. Each section 8 contains a part of the superabsorbent material included in the sheet 5, the material in one section being kept apart from the material in adjoining sections. This means that fluid delivered to a bag in which the sheet is arranged converts the superabsorbent material in the sheet to a gel composition in one section after another, starting in the section located at the bottom of the bag. This further ensures that no more superabsorbent material is used than is actually needed on each occasion. The sections 8 can be separated from one another by the surface layers having been joined and bonded to one another along transverse connection lines 9.

As has been described above, the laminate sheets 5 are punched out from a relatively stiff blank. There is a risk here of particles from the superabsorbent layer 7 between the surface layers 6 coming loose. These particles can then settle on areas of the collection bag which are to be welded together during its production. If this happens, it may result in weld seams that leak. It is therefore preferred that the side edges of each laminate sheet 5 are sealed together.

According to the present invention, this can be done by moistening the side edges of the sheet 5, for example with the aid of vapor, so that the outermost superabsorbent material 7 located between the surface layers 6 is converted to a gel strand 10 (see FIG. 4) which closes the edge portions. This closure can also contribute to preventing urine that runs down along the surface layers 6 of the sheet 5 from penetrating into the superabsorbent material between these on its way down to the lower portion of the sheet.

FIG. 5 illustrates another embodiment of a device according to the invention comprising a catheter bag 11 intended to collect urine delivered to the bag via a line 12. Reference number 13 designates a simple nonreturn valve mechanism consisting of two film portions bearing against one another. A superabsorbent laminate sheet 5 of the previously described configuration is arranged in the catheter bag 11.

A catheter bag of this kind can, for example, be secured on a leg. For the user, it is a great advantage that urine delivered to the bag is converted directly to a gel composition with the aid of the superabsorbent sheet 5 arranged in the bag in a manner corresponding to that described above.

The invention has been described above in connection with the embodiments shown in the drawings. However, a device according to the invention can be varied in several respects within the scope of the patent claims, among other things for adaptation to other applications in which body fluid is to be collected. For example, any desired material for the surface layers can be chosen as long as it has the characteristic specified in the patent claims. The superabsorbent sheets can be produced not just by rolling, but also by pressing.

1. A device for collecting urine or other organic body fluids, comprising a flexible bag (1; 11) made of liquid-impermeable film material, a member (2; 12) for receiving fluid and conveying it into the bag, means (13) for preventing fluid from leaving the bag via said receiving member, and at least one absorption body arranged in the bag (1) and in the form of a sheet-shaped laminate (5) comprising a superabsorbent material (7) which is enclosed between two liquid-permeable surface layers (6) and which forms a gel composition (14) when delivered fluid is absorbed, characterized in that the surface layers (6) of the laminate sheet (5) have such a porosity that delivered fluid in brief contact with a surface layer of a non-horizontal sheet (5) will run down the outside of the latter without penetrating to any appreciable extent through the surface layer (6), whereas delivered fluid which forms a fluid collection in contact with a surface layer of the sheet penetrates through the latter so as to be absorbed by the superabsorbent material (7) located to the inside of the surface layer and so as to convert this superabsorbent material (7) to a gel composition (14).

2. The device as claimed in claim 1, characterized in that the laminate sheet (5) is supported by the bottom of the bag (1), in that the surface layers (6) of the sheet are made of a flexible material which allows the laminate sheet to collapse toward the bottom of the bag as the superabsorbent material (7) in the sheet is converted to a gel composition (14) by delivered fluid.

3. The device as claimed in claim 1, characterized in that the surface layers (6) of the laminate sheet (1) are made of compressed tissue material.

4. The device as claimed in claim 3, characterized in that the surface layers (6) of compressed tissue material have such a pore size that fluid in brief contact with a surface layer does not pass through the latter.

5. The device as claimed in claim 4, characterized in that the surface layers (6) of tissue material are rolled together with an intermediate layer of superabsorbent material (7) in powder form, using a roll pressure which produces the desired pore size.

6. The device as claimed in claim 3, characterized in that the tissue material in the surface layers (6) is compressed to a density of 0.7 g/cm³±20%.

7. The device as claimed in claim 3, characterized in that the tissue material constitutes 2-6 percent by weight of the laminate sheet.

8. The device as claimed in claim 3, characterized in that the tissue material has a pore size of 200 μm±100 μm.

9. The device as claimed in claim 1, characterized in that the edge portions of the laminate sheet (1) are closed with a gel strand (10) comprising superabsorbent material (7) converted to gel and arranged between the edge portions of the surface layers (6).

10. The device as claimed in claim 1, characterized in that the laminate sheet (5) is divided into sections (8) from the lower end portion of the sheet and upward, in that each section (8) contains part of the superabsorbent material (7) included in the sheet, in that the material in one section is kept separate from the material in adjoining sections so that fluid delivered to the bag (1; 11) converts the superabsorbent material (7), in a sheet (5) supported by the bottom of the bag, to a gel composition (14) one section after another, starting in the section located at the bottom of the bag.

11. A laminate sheet of the type included in the device as claimed in claim 1.

12. The laminate sheet as claimed in claim 11, characterized in that it contains 30 g±5 g of superabsorbent material (7).

13. A method for producing superabsorbent laminate sheets in which two layers of tissue material and an intermediate layer of superabsorbent material are rolled together to form a blank from which laminate sheets of the desired size are punched out, characterized in that a roll pressure is used which gives the blank such stiffness that the laminate sheets punched out from it are self-supporting and can be placed on the bottom of a collection bag intended for receiving urine or other organic body fluids, and which roll pressure compresses the tissue material in the surface layers of the laminate sheets in order to reduce the porosity of these to such a degree that fluid in brief contact with a surface layer of a non-horizontal sheet will run down the outside of the latter without penetrating to any appreciable extent through the surface layer, whereas delivered fluid which forms a fluid collection in contact with a surface layer of the sheet penetrates through the latter so as to be absorbed b the superabsorbent material located to the inside of the surface layer and so as to convert this superabsorbent material to a gel composition.

14. The method as claimed in claim 13, characterized in that the tissue material in the surface layers of the laminate sheets in compressed to a density of 0.7 g/cm³±20%.

15. The method as claimed in claim 13, characterized in that the tissue material is compressed to such an extent that it acquires a pore size of 200 μm+100 μm.

16. The method as claimed in claim 13, characterized in that at least the side edges of the punched-out laminate sheets are moistened so that the superabsorbent material located between the edge portions of the surface layers is converted to a gel composition closing the side edges of the laminate sheets.

17. The method as claimed in claim 16, characterized in that said side edges are moistened with the aid of water vapor.

18. The method as claimed in of claim 13, characterized in that the superabsorbent material in the laminate sheet is divided into mutually separate sections, from the lower end portion of the sheet upward.

19. The method as claimed in claim 18, characterized in that the division of the laminate sheet is effected by the surface layers being joined and bonded to one another along transverse connection lines.
